(19) United States
(12) Patent Application Publication
(10) Pub. No.: US 2019/0105197 A1
(43) Pub. Date: Apr. 11, 2019

(54) DEVICES AND METHODS FOR INJECTION BENEATH EYE TISSUE

(71) Applicant: Novartis AG, Basel (CH)

(72) Inventors: Mark David LaBelle, Mattoon, IL (US); Brian William McDonell, Irvine, CA (US)

(21) Appl. No.: 16/130,330

(22) Filed: Sep. 13, 2018

Related U.S. Application Data
(60) Provisional application No. 62/568,382, filed on Oct. 5, 2017.

(51) Int. Cl. A61F 9/00; A61M 5/32

(52) U.S. Cl. CPC A61F 9/0017; A61F 9/00736; A61M 5/32

(57) ABSTRACT

Devices and methods are provided for injection of a therapeutic agent into an area beneath eye tissue, such as a sub-retinal area or a suprachoroidal area. Example devices comprise an injector housing, a tubular element (needle or microcatheter), a first area within the injector housing configured to be selectively in fluid communication with the tubular element for delivery of a bleb-formation fluid, and a second area within the injector housing configured to be selectively in fluid communication with the tubular element for delivery of a therapeutic agent into the area beneath eye tissue. In some embodiments, the first area is a chamber within the injector housing, and the second area is an internal area of a tube within the chamber. The devices and methods allow, with a single needle (or microcatheter) insertion, high pressure bleb formation and low pressure delivery of fragile therapeutic agent compositions.

This application claims the benefit of priority of U.S. Provisional Patent Application Ser. No. 62/568,382 titled “DEVICES AND METHODS FOR INJECTION BENEATH EYE TISSUE”, filed on Oct. 5, 2017, whose inventors are Mark David LaBelle and Brian William McDonell, which is hereby incorporated by reference in its entirety as though fully and completely set forth herein.

### TECHNICAL FIELD

The present disclosure is directed to medical devices and methods for the treatment of ocular conditions by the injection of a therapeutic agent into an area beneath eye tissue, such as beneath retinal tissue in a sub-retinal space or beneath choroidal tissue in a suprachoroidal space.

### BACKGROUND

A number of ocular conditions have been treated by the injection of one or more therapeutic agents into the eye. In recent years, the use of live cell vectors, viral therapeutic agents, gene therapy vectors, stem cells, and other relatively delicate therapeutic agents has been proposed for the treatment of various eye conditions. For example, such therapeutic agents have been proposed for treatment of such conditions as macular degeneration, Leber's congenital amaurosis, Stargardt's disease, and other conditions.

For certain conditions and therapeutic agents, it is desirable to create a sub-retinal pocket or bleb beneath the retinal tissue and subsequently to deliver the therapeutic agent into this volume. However, the pressures and/or flow rates required for pocket or bleb formation can be detrimental to delicate therapeutic agents. A need exists for improved devices and methods for the delivery of delicate therapeutic agents beneath eye tissue.

### SUMMARY

The present disclosure is directed to improved devices and methods for injections of therapeutic agent beneath eye tissue, such as beneath retinal tissue in a sub-retinal space or beneath choroidal tissue in a suprachoroidal space.

Exemplary devices and methods are provided herein. One general aspect according to some embodiments includes a device for injection of therapeutic agent into an area beneath eye tissue, the device comprising: an injector housing having a proximal end and a distal end and an outlet port at the distal end; a tubular element (such as a needle or catheter) having a lumen connected to the outlet port of the injector housing at the distal end of the injector housing; a first area within the injector housing configured to be selectively in fluid communication with the tubular element for delivery of a bleb-formation fluid through the tubular element into the area beneath eye tissue for formation of a bleb beneath the eye tissue; and a second area within the injector housing configured to be selectively in fluid communication with the tubular element for delivery of a therapeutic agent through the tubular element into the area beneath the eye tissue at a location of the bleb; wherein the device comprises a first condition in which the first area is in fluid communication with the tubular element for delivery of the bleb-formation fluid through the tubular element into the area beneath eye tissue and a second condition in which the second area is in fluid communication with the tubular element for delivery of the therapeutic agent through the tubular element into the area beneath eye tissue. The therapeutic agent may comprise at least a live cell vector, viral therapeutic agent, a gene therapy vector, or stem cells.

In further aspects according to some embodiments, when the device is in the first condition, fluid communication between the second area and the tubular element is prevented, and when the device is in the second condition, fluid communication between the first area and the tubular element is prevented.

In some embodiments, the first area is a chamber within the injector housing and the second area is an internal area of a tube within the chamber, wherein the tube is movable between a proximal position in which it is away from the outlet port of the injector housing and a distal position in which it is adjacent the outlet port of the injector housing. When the tube is in the proximal position the device is in the first condition in which the first area is in fluid communication with the tubular element for delivery of the bleb-formation fluid through the tubular element, and when the tube is in the distal position the device is in the second condition in which the second area is in fluid communication with the tubular element for delivery of the therapeutic agent through the tubular element.

In alternative embodiments, the second area is a chamber within the injector housing and the first area is an internal area of a tube within the chamber, wherein the tube is movable between a proximal position in which it is away from the outlet port of the injector housing and a distal position in which it is adjacent the outlet port of the injector housing. When the tube is in the distal position the device is in the first condition in which the first area is in fluid communication with the tubular element for delivery of the bleb-formation fluid through the tubular element, and when the tube is in the proximal position the device is in the second condition in which the second area is in fluid communication with the tubular element for delivery of the therapeutic agent through the tubular element.

An example embodiment of a method for injection of therapeutic agent into an area beneath eye tissue comprises: (i) advancing a tip of a tubular element of an injector device into the area beneath eye tissue, (ii) with the device in a first condition in which a first area is in fluid communication with the tubular element, delivering bleb-formation fluid from the first area through the tubular element into the area beneath eye tissue, (iii) switching the device from the first condition to a second condition in which the second area is in fluid communication with the tubular element, and (iv) with the device in the second condition, delivering the therapeutic agent through the tubular element into the area beneath eye tissue.

Devices and methods in accordance with certain embodiments as described herein facilitate delivery of the bleb-formation fluid into the area beneath eye tissue, such as a sub-retinal area or suprachoroidal area, at a higher pressure than the delivery of the therapeutic agent. In some embodiments, the therapeutic agent may be delivered without exposing the therapeutic agent to the higher pressure of the bleb-formation fluid.

Devices and methods in accordance with certain embodiments as described herein facilitate delivery of a bleb-formation fluid into an area beneath eye tissue and delivery of a therapeutic agent into that area with only one puncture of eye tissue into the desired area and with minimal motion of the tubular element (needle or catheter) throughout the process. Once the tissue is punctured, all actions of the injector can be controlled remotely and can be initiated quickly. In some embodiments, no motion of the hand holding the device is required to actuate the delivery of the bleb-formation fluid and the therapeutic agent.

It is to be understood that both the foregoing general description and the following detailed description are exemplary and explanatory in nature and are intended to provide an understanding of the disclosure without limiting the scope of the disclosure. In that regard, additional aspects, features, and advantages of the disclosure will be apparent to one skilled in the art from the accompanying drawings and the following detailed description.

### BRIEF DESCRIPTION OF THE DRAWINGS

The accompanying drawings illustrate implementations of the devices and methods disclosed herein and, together with the description, serve to explain the principles of the disclosure.

FIG. 1 illustrates a perspective view of an exemplary device for injection beneath eye tissue according to aspects of the present disclosure.

FIG. 2 is a schematic illustration of a cross-section of part of the injector housing of FIG. 1, according to an embodiment.

FIG. 3A is a cross-sectional illustration of another exemplary device for injection beneath eye tissue according to aspects of the present disclosure, with an internal tube in a proximal position, according to an embodiment.

FIG. 3B is a cross-sectional illustration of the device of FIG. 3A, with an internal tube in a distal position, according to an embodiment.

FIGS. 4A and 4B are an end view and a cross-sectional side view, respectively, of a guide that is a part of the device of FIGS. 3A and 3B, according to an embodiment.

FIGS. 5A and 5B are an end view and a cross-sectional side view, respectively, of a valve that is a part of the device of FIGS. 3A and 3B, according to an embodiment.

FIG. 6A is a cross-sectional illustration of another exemplary device for injection beneath eye tissue according to aspects of the present disclosure, with an internal tube in a proximal position.

FIG. 6B is a cross-sectional illustration of the device of FIG. 6A, with an internal tube in a distal position, according to an embodiment.

FIG. 7 illustrates a flowchart of a method for delivery of a bleb-formation fluid into an area beneath eye tissue and delivery of a therapeutic agent into that area, according to an embodiment.

The accompanying drawings may be better understood by reference to the following detailed description.

### DETAILED DESCRIPTION

For the purposes of promoting an understanding of the principles of the present disclosure, reference will now be made to the implementations illustrated in the drawings, and specific language will be used to describe the same. It will nevertheless be understood that no limitation of the scope of the disclosure is intended. Any alterations and further modifications to the described devices, instruments, and methods, and any further application of the principles of the present disclosure, are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure. For simplicity, in some instances the same reference numbers are used throughout the drawings to refer to the same or like parts.

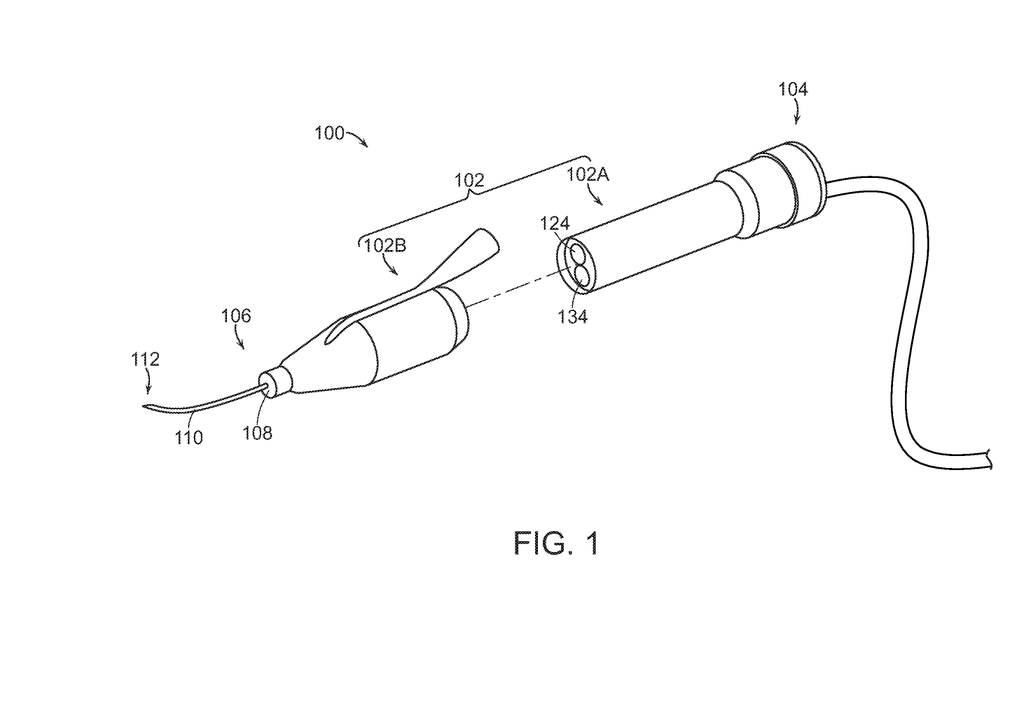

FIG. 1 illustrates a perspective view of an exemplary device 100 for injection beneath eye tissue according to aspects of the present disclosure.

As shown in FIG. 1, the device 100 comprises an injector housing 102 that is in two parts, proximal part 102A and distal part 102B. The two parts can be connected together in any suitable manner, for example by screw thread connection, snap fit, Luer lock, and other suitable connections. The overall injector housing 102 has a proximal end 104 and a distal end 106. The injector housing 102 has an outlet port 108 at the distal end 106. The outer shape of the injector housing 102 may be any suitable shape, such as square, circular, elliptical, etc.

A tubular element which in this example is a rigid needle 110 is connected to the outlet port 108 of the injector housing 102 at the distal end 106 of the injector housing 102. In this example, the distal end of the needle 110 is curved to facilitate access beneath eye tissue, such as beneath retinal tissue into a sub-retinal area. The needle 110 terminates in a needle tip 112.

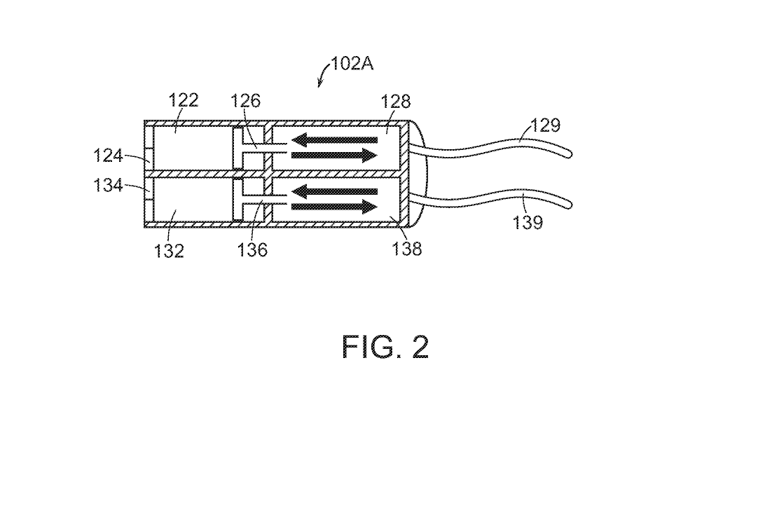

FIG. 2 is a schematic illustration of a cross-section of the proximal part 102A of the injector housing 102 of FIG. 1. The internal area of the injector housing 102 has at least two separate areas or compartments which hold, or through which may be delivered, the intended fluids or therapeutic agent compositions for delivery. Thus, for example, as shown in FIG. 2, the device 100 comprises a first area 122 within the injector housing 102 and a second area 132 within the injector housing 102.

The first area 122 and the second area 132 in this example are in the form of chambers that are sealed at their distal ends by valves 124 and 134, respectively. Pistons 126, 136, shown schematically in FIG. 2, enter the proximal ends of the first and second areas 122, 132, respectively. The entry of the pistons 126, 136 into the first and second areas 122, 132 is sealed in order to prevent any escape or leakage of fluid or therapeutic agent from the first and second areas 122, 132.

The pistons 126, 136 are attached to suitable motors or driving mechanisms 128, 138, respectively. The driving mechanisms may be, for example, any suitable motor or drive, such as a stepper motor, pneumatic drive, hydraulic drive, screw drive, or other mechanism for driving the piston to generate the appropriate and desired speed and pressure of delivery. The delivery mechanisms 128, 138 may be activated by an operator via electrical control lines 129, 139, respectively, that are connected, for example, to a foot pedal or other actuation device.

In one example, a bleb-formation fluid may be delivered from the first area 122, and a therapeutic agent composition may be delivered from the second area 132. This may be reversed, such that a bleb-formation fluid is delivered from the area 132, and a therapeutic agent composition is delivered from the area 122. The first and second areas 122, 132 may be prefilled before the medical procedure. In an example, the first and second areas 122, 132 may comprise or be loaded with cartridges that are prefilled. Alternatively, supply lines may be provided to the first area 122 and/or the second area 132 to supply the desired fluid or therapeutic agent composition to the first area 122 and/or the second area 132 before or during the medical procedure. A source of bleb-formation fluid or therapeutic agent composition may be provided at the proximal end of the supply line, and the source may be pressurized or driven by a suitable delivery mechanism as described above. A valve such as a check valve may be provided where the supply line meets the respective first or second area 122, 132 to prevent backflow.

The valves 124 and 134 at the distal ends of the first area 122 and the second area 132 may be one-way valves that allow flow from the respective areas 122, 132 but not into the respective areas 122, 132. The valves 124, 134 may be designed to open only upon the application of a certain pressure, exerted on the contents of the areas 122, 132 by the respective pistons 126, 136 or by a pressurization mechanism on the fluid through the supply line.

Thus, for example, when the driving mechanism 128 is activated and the piston 126 is driven, the resulting pressure on the contents of the first area 122 causes the valve 124 to open, allowing the contents to be delivered distally, through the outlet port 108 and the needle 110. Similarly, when the driving mechanism 138 is activated and the piston 136 is driven, the resulting pressure on the contents of the second area 132 causes the valve 134 to open, allowing the contents to be delivered distally, through the outlet port 108 and the needle 110. In this manner, each of the first area 122 and the second area 132 is configured to be selectively in fluid communication with the needle 110.

In an example in which a bleb-formation fluid is delivered from the first area 122 and a therapeutic agent composition is delivered from the second area 132, the device 100 may be characterized as comprising a first condition in which the first area 122 is in fluid communication with the needle 110 for delivery of the bleb-formation fluid through the needle 110 and a second condition in which the second area 132 is in fluid communication with the needle 110 for delivery of the therapeutic agent through the needle 110. When the device 100 is in the first condition, in which pressure is being exerted on the bleb-formation fluid, fluid communication between the second area 132 and the needle 110 is prevented. In this example, this is due to the valve 134 remaining closed. When the device 100 is in the second condition, in which pressure is being exerted on the therapeutic agent composition, fluid communication between the first area 122 and the needle 110 is prevented. In this example, this is due to the valve 124 remaining closed.

As an example, the bleb-formation fluid may be a balanced saline solution. The therapeutic agent may comprise, for example, a live cell vector, viral therapeutic agent, a gene therapy vector, and/or stem cells.

The device 100 and the pressurization or driving mechanisms may be designed to deliver the bleb-formation fluid at a first pressure suitable for bleb formation and to deliver the therapeutic agent composition at a lower pressure suitable for the therapeutic agent. The therapeutic agent, particularly live cell vectors, viral therapeutic agents, gene therapy vectors, and/or stem cells, may be delicate and could be negatively affected by high pressure delivery. Accordingly, the device 100 allows for the bleb formation to be conducted at a separate and higher pressure than the therapeutic agent delivery.

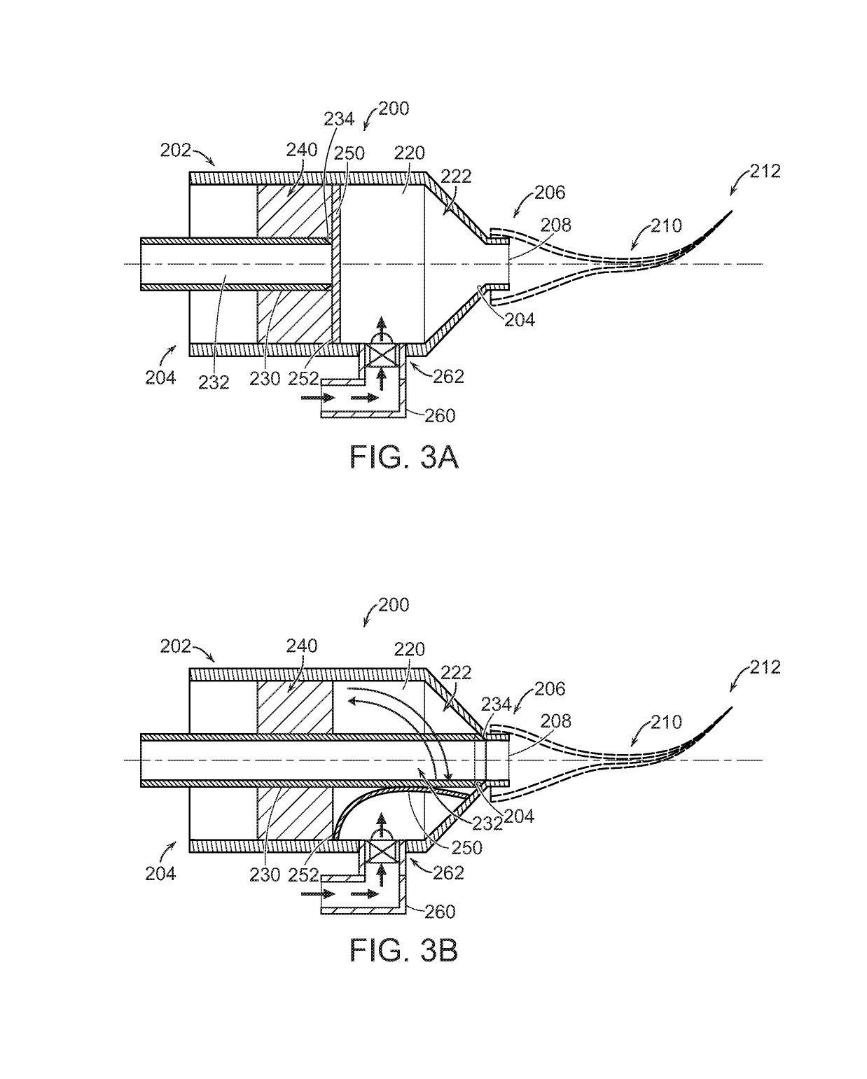

FIGS. 3A and 3B are cross-sectional illustrations of another exemplary device 200 for injection beneath eye tissue according to aspects of the present disclosure.

The device 200 comprises an injector housing 202 that, like the device 100, may be a multi-part or a single part injector housing. The injector housing 202 has a proximal end 204 and a distal end 206. The injector housing 202 has an outlet port 208 at the distal end 206.

A tubular element which in this example is a rigid needle 210 is connected to the outlet port 208 of the injector housing 202 at the distal end 206 of the injector housing 202. As with needle 110, the distal end of the needle 210 is curved to facilitate access beneath eye tissue, such as beneath retinal tissue into a sub-retinal area. The needle 210 terminates in a needle tip 212.

The internal area of the injector housing 202 has at least two separate areas or compartments which hold, or through which may be delivered, the intended fluids or therapeutic agent compositions for delivery. Thus, for example, as shown in FIGS. 3A and 3B, the device 200 comprises a first area 222 within the injector housing 202 and a second area 232 within the injector housing 202.

In this example, the first area 222 is a chamber 220 within the injector housing 202, and the second area 232 is an internal area of a tube 230 within the chamber 220. The tube 230 is movable between a proximal position in which it is away from the outlet port 208 of the injector housing 202 and a distal position in which it is adjacent the outlet port 208 of the injector housing 202. The proximal position of the tube 230 is shown in FIG. 3A. The distal position of the tube 230 is shown in FIG. 3B.

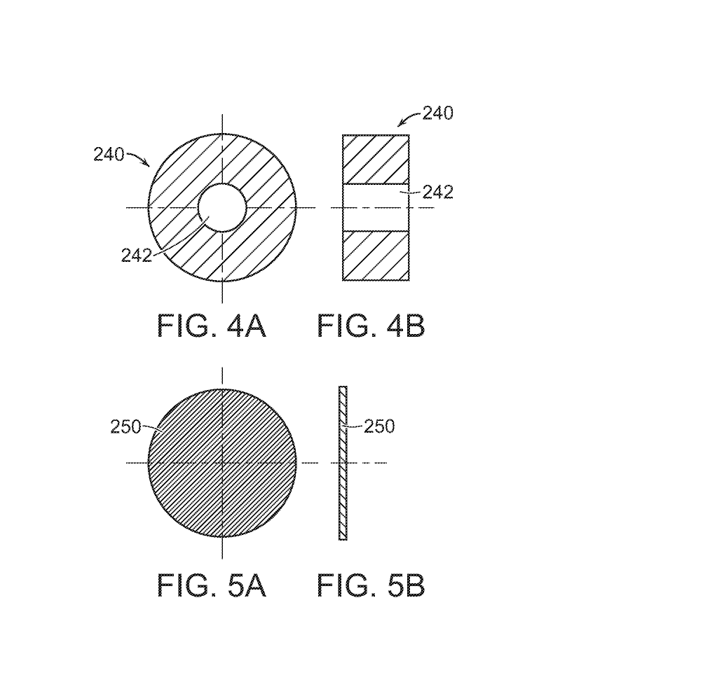

In order to stabilize the tube 230 and guide it within the chamber 220, the device 200 comprises a guide element 240, shown further in FIGS. 4A and 4B. The guide element 240 is sized and shaped to fit securely within the injector housing 202. The guide element has an internal bore 242 sized and shaped to securely accommodate the tube 230, allowing the tube to be moved longitudinally with respect to the guide element 240. The guide element 240 may be a separate piece or integrally formed with the injector housing 202.

In one version, when the tube 230 is in the proximal position, the device 200 is in the first condition in which the first area 222 is in fluid communication with the needle 210 for delivery of the bleb-formation fluid through the needle 210. When the tube 230 is moved to the distal position, the device 200 is in the second condition in which the second area 232 is in fluid communication with the needle 210 for delivery of the therapeutic agent through the needle 210.

In an alternative version, the areas are reversed. That is, the area 232 serves as the first area for delivery of the bleb-formation fluid, and the area 222 serves as the second area for delivery of the therapeutic agent. In this variation, when the tube 230 is in the distal position, the device 200 is in the first condition in which the area 232 (now the first area) is in fluid communication with the needle 210 for delivery of the bleb-formation fluid through the needle 210. When the tube 230 is then moved to the proximal position, the device 200 is in the second condition in which the area 222 (now the second area) is in fluid communication with the needle 210 for delivery of the therapeutic agent through the needle 210.

In some embodiments, the first area 222 and the second area 232 may be sealed at their distal ends by one-way valves in a similar manner as described above. Pistons may enter the proximal ends of the first and/or second areas 222, 232, respectively, in a sealed manner in order to prevent any escape or leakage of fluid or therapeutic agent from the first and/or second areas 222, 232. The pistons may be attached to suitable motors or driving mechanisms, which may be any of the driving mechanisms described above. The delivery mechanisms may be activated by an operator via electrical control lines that are connected, for example, to a foot pedal or other actuation device.

In the example shown in FIGS. 3A and 3B, the device 200 comprises a reed valve 250. The reed valve 250 is further shown in FIGS. 5A and 5B. The reed valve 250 seals the distal or outlet end of the tube 230 and area 232 when the tube 230 is in the proximal position, shown in FIG. 3A. The reed valve 250 is designed to allow opening in only the distal direction. Thus, when the tube 230 is advanced distally, the reed valve 250 opens. For example, the reed valve 250 may be secured on one side, for example at location 252. When the tube 230 is in the distal position, the reed valve 250 is forced to its open position, shown in FIG. 3B.

In one example manner of use of device 200, a bleb-formation fluid may be delivered from the first area 222, and a therapeutic agent composition may be delivered from the second area 232. This may be reversed, such that a bleb-formation fluid is delivered from the area 232, and a therapeutic agent composition is delivered from the area 222. The first and second areas 222, 232 may be prefilled before the medical procedure. In an example, the first and second areas 222, 232 may comprise or be loaded with cartridges that are prefilled. Alternatively, supply lines may be provided to the first area 222 and/or the second area 232 to supply the desired fluid or therapeutic agent composition to the first area 222 and/or the second area 232 before or during the medical procedure. A source of bleb-formation fluid or therapeutic agent composition may be provided at the proximal end of the supply line, and the source may be pressurized or driven by a suitable delivery mechanism as described above. A valve such as a check valve may be provided where the supply line meets the respective first or second area 222, 232 to prevent backflow. FIGS. 3A and 3B show an example supply line 260 having a check valve 262.

In this example, when the tube 230 is in the proximal position, as shown in FIG. 3A, the chamber 220 and area 222 are in fluid communication with the needle 210. When the tube 230 is in the distal position, as shown in FIG. 3B, the tube 230 and area 232 are in fluid communication with the needle. When one of the areas 222, 232 is in fluid communication with the needle 210, flow from the other area 232, 222 is prevented. Thus, for example, when the tube 230 is in the proximal position and the area 222 is in fluid communication with the needle 210, the valve 250 prevents flow from or into the area 232. When the tube 230 is in the distal position, the leading edge 234 of the tube engages a shoulder 204 of the injector housing 202 adjacent the outlet port 208 to create a seal. In this manner, flow from the area 222 is prevented. In this manner, each of the first area 222 and the second area 232 is configured to be selectively in fluid communication with the needle 210 while flow from the other area 232, 222 is prevented.

In an example in which a bleb-formation fluid is delivered from the first area 222 and a therapeutic agent composition is delivered from the second area 232, the device 200 may be characterized as comprising a first condition in which the first area 222 is in fluid communication with the needle 210 for delivery of the bleb-formation fluid through the needle 210 and a second condition in which the second area 232 is in fluid communication with the needle 210 for delivery of the therapeutic agent through the needle 210. When the device 200 is in the first condition, fluid communication between the second area 232 and the needle 210 is prevented. In this example, this is due to the valve 250 remaining closed. When the device 200 is in the second condition, fluid communication between the first area 222 and the needle 210 is prevented. In this example, this may be due to the seal between edge 234 and shoulder 204.

The device 200 is advantageous in minimizing dead space in the internal volume. For example, the tube 230 may be filled with a therapeutic agent composition to its outlet end. When the tube 230 is advanced to its distal position, the outlet end of the tube 230 is adjacent the outlet port 208 of the injector housing. Thus, the space the therapeutic agent composition must travel to reach the needle 210 is minimized.

In some embodiments, the outlet end of the tube 230 may be covered with a septum that can be pierced. In such a case, the septum prevents delivery from the tube, and a separate valve may not be needed. When the tube 230 is advanced to the distal position, the septum engages a piercing member or edge at the shoulder 204 of the injector housing 202, causing the septum to be pierced. This then puts the tube 230 in fluid communication with the needle 210.

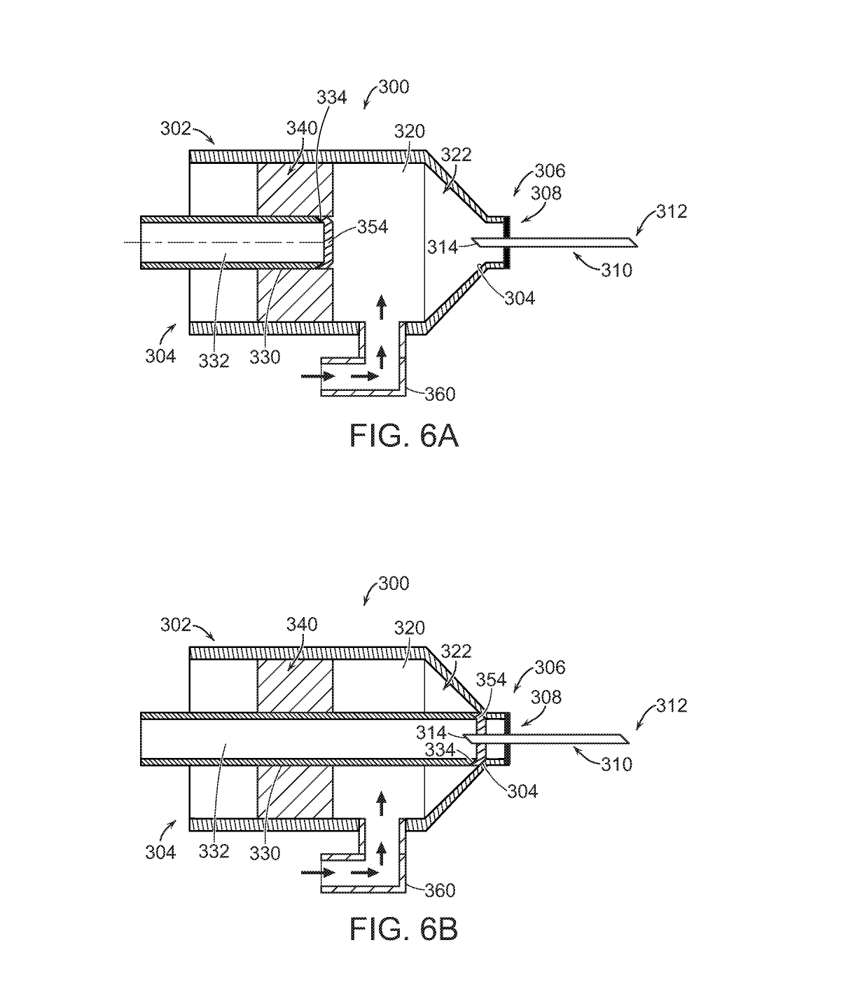

Another example of a device with a septum that may be pierced is shown in FIGS. 6A and 6B, which are cross-sectional illustrations of another exemplary device 300 for injection beneath eye tissue according to aspects of the present disclosure.

The device 300 comprises an injector housing 302 that, like the devices 100 and 200, may be a multi-part or a single part injector housing. The injector housing 302 has a proximal end 304 and a distal end 306. The injector housing 302 has an outlet port 308 at the distal end 306.

A tubular element which in this example is a rigid needle 310 is connected to the outlet port 308 of the injector housing 302 at the distal end 306 of the injector housing 302. As with needle 110, the distal end of the needle 310 is curved to facilitate access beneath eye tissue, such as beneath retinal tissue into a sub-retinal area. The needle 310 terminates at its distal end in a needle tip 312. At its proximal end, the needle 310 has a sharp piercing member or tip 314 which extends into the injector housing 302.

The internal area of the injector housing 302 has at least two separate areas or compartments which hold, or through which may be delivered, the intended fluids or therapeutic agent compositions for delivery. Thus, for example, as shown in FIGS. 6A and 6B, the device 300 comprises a first area 322 within the injector housing 302 and a second area 332 within the injector housing 302.

In this example, the first area 322 is a chamber 320 within the injector housing 302, and the second area 332 is an internal area of a tube 330 within the chamber 320. The tube 330 is movable between a proximal position in which it is away from the outlet port 308 of the injector housing 302 and a distal position in which it is adjacent the outlet port 308 of the injector housing 302. The proximal position of the tube 330 is shown in FIG. 6A. The distal position of the tube 330 is shown in FIG. 6B.

In order to stabilize the tube 330 and guide it within the chamber 320, the device 300 comprises a guide element 340, similar to the guide element 240. The guide element 340 is sized and shaped to fit securely within the injector housing 302. The guide element has an internal bore (like bore 242 shown in FIGS. 4A and 4B) sized and shaped to securely accommodate the tube 330, allowing the tube to be moved longitudinally with respect to the guide element 340. The guide element 340 may be a separate piece or integrally formed with the injector housing 302.

In one version, when the tube 330 is in the proximal position, the device 300 is in the first condition in which the first area 322 is in fluid communication with the needle 310 for delivery of the bleb-formation fluid through the needle 310. When the tube 330 is moved to the distal position, the device 300 is in the second condition in which the second area 332 is in fluid communication with the needle 310 for delivery of the therapeutic agent through the needle 310.

In the example shown in FIGS. 6A and 6B, the outlet end of the tube 330 is covered with a septum 354 that can be pierced. When intact, as shown in FIG. 6A, the septum 354 prevents delivery from the tube 330. When the tube 330 is advanced to the distal position, as shown in FIG. 6B, the septum 354 is forced onto the piercing member or needle tip 314, which pierces the septum 354. This then puts the tube 330 and second area 332 in fluid communication with the needle 310.

In one example manner of use of device 300, a bleb-formation fluid may be delivered from the first area 322, and a therapeutic agent composition may be delivered from the second area 332. The first and second areas 322, 332 may be prefilled before the medical procedure. In an example, the first and second areas 322, 332 may comprise or be loaded with cartridges that are prefilled. For example, a cartridge (or vial) with the therapeutic agent composition may be loaded into the second area 332 before use of the device 300. The cartridge may have a septum at its distal end which, like septum 354, is pierceable by the piercing member 314 when the tube 330 is advanced to the distal position (as described below). In such a case, the septum of the cartridge can be in addition to or in replacement of the septum of the tube 330. Alternatively, supply lines may be provided to the first area 322 and/or the second area 332 to supply the desired fluid or therapeutic agent composition to the first area 322 and/or the second area 332 before or during the medical procedure. A source of bleb-formation fluid or therapeutic agent composition may be provided at the proximal end of the supply line, and the source may be pressurized or driven by a suitable delivery mechanism as described above. As just one possible example, the bleb-formation fluid is supplied through the first area 322 from a pressurized source through supply line 360, while the therapeutic agent composition may be delivered from the second area 332 by a pressurized source at the proximal end of the tube 330 or by a piston within the tube 330. The delivery mechanisms may be activated by an operator via electrical control lines that are connected, for example, to a foot pedal or other remote actuation device.

In this example, when the tube 330 is in the proximal position, as shown in FIG. 6A, the chamber 320 and first area 322 are in fluid communication with the needle 310. When the tube 330 is in the distal position, as shown in FIG. 6B, the tube 330 and second area 332 are in fluid communication with the needle 310. When one of the areas 322, 332 is in fluid communication with the needle 310, flow from the other area 332, 322 is prevented. Thus, for example, when the tube 330 is in the proximal position and the first area 322 is in fluid communication with the needle 310, the septum 354 prevents flow from or into the second area 332. When the tube 330 is in the distal position, the leading edge 334 of the tube engages a shoulder 304 of the injector housing 302 adjacent the outlet port 308. The septum 354 is forced onto the piercing member 314, which pierces the septum 354, putting the second area 332 in fluid communication with the needle 310. In this manner, each of the first area 322 and the second area 332 is configured to be selectively in fluid communication with the needle 310 while flow from the other area 332, 322 is prevented.

In an example in which a bleb-formation fluid is delivered from the first area 322 and a therapeutic agent composition is delivered from the second area 332, the device 300 may be characterized as comprising a first condition in which the first area 322 is in fluid communication with the needle 310 for delivery of the bleb-formation fluid through the needle 310 and a second condition in which the second area 332 is in fluid communication with the needle 310 for delivery of the therapeutic agent through the needle 310. When the device 300 is in the first condition, fluid communication between the second area 332 and the needle 310 is prevented. In this example, this is due to the septum 354 covering and closing the end of the tube 330. When the device 300 is in the second condition, fluid communication between the first area 322 and the needle 310 is prevented. In this example, this may be due to the proximal end of the needle 310 having entered the tube 330 through the septum 354 such that the first area 322 is no longer in fluid communication with the proximal end of the needle 310.

The device 300 is advantageous in minimizing dead space in the internal volume. For example, the tube 330 may be filled with a therapeutic agent composition to its outlet end. When the tube 330 is advanced to its distal position, the outlet end of the tube 230 is in direct fluid communication with the needle 310. Thus, the space the therapeutic agent composition must travel to reach the needle 310 is minimized.

The devices 100, 200, 300 and other devices in accordance with the principles described herein may be used advantageously to deliver delicate therapeutic agents into an area beneath eye tissue, such as a sub-retinal space or a suprachoroidal space.

In certain embodiments, the tubular element, such as tubular element 110, 210, or 310 may be a flexible microcatheter. This may be, for example, for use of the device in an ab externo procedure for accessing a sub-retinal space or a suprachoroidal space.

In an example procedure, a physician directs the tubular element 110, 210, 310 into the eye in a manner as known in the art and advances the tubular element tip 112, 212, 312 beneath the desired eye tissue in order to access the target area beneath eye tissue. For example, in an ab interno procedure, the physician directs the tubular element 110, 210, 310, which may be a needle, into the posterior segment of the eye and advances the needle tip 112, 212, 312 into and beneath the retina in order to access a sub-retinal area. As another example, in an ab interno procedure, the physician directs the needle 110, 210, 310 into the posterior segment of the eye and advances the needle tip 112, 212, 312 into and beneath the choroid in order to access a suprachoroidal area.

As another possible example, in an ab externo procedure, the physician directs the tubular element 110, 210, 310, which may be a microcatheter, through the sclera (through an access opening previously formed by a sclerotomy) and advances the tubular element tip 112, 212, 312 beneath the choroid in order to access a suprachoroidal area. As another example, in an ab externo procedure, the physician directs the tubular element 110, 210, 310, which may be a microcatheter, through the sclera (through an access opening previously formed in the sclera by a sclerotomy) and through the choroid (through an access opening previously formed in the choroid by a choroidotomy) and advances the tubular element tip 112, 212, 312 beneath the retina in order to access a sub-retinal area.

With the device 100, 200, 300 in a first condition in which the first area 122, 222, 322 is in fluid communication with the tubular element (needle or microcatheter), the physician then delivers the bleb-formation fluid (e.g., balanced saline solution) through the tubular element 110, 210, 310 into the target area beneath eye tissue, e.g., the sub-retinal area or suprachoroidal area. This may be done by operating a foot pedal or other remote actuation device. High pressures may be used to facilitate bleb formation in the sub-retinal or suprachoroidal space. When the device 100, 200, 300 is in the first condition, fluid communication between the second area 132, 232, 332 and the tubular element 110, 210, 310 is prevented.

When the bleb is sufficiently formed in the sub-retinal or suprachoroidal area, the physician then switches the device 100, 200, 300 from the first condition to a second condition in which the second area is in fluid communication with the tubular element. This may be done simply by deactivating pressure on the first area 122 and activating pressure on the second area 132. Alternatively, in an example as in FIGS. 3A and 3B and 6A and 6B, this may be done by advancing a tube 230, 330 from a proximal position to a distal position, or, as a variant of the embodiment of FIGS. 3A and 3B, by retracting a tube 230 from a distal position to a proximal position. These actions may be done by operating a foot pedal or other remote actuation device.

With the device 100, 200, 300 in the second condition, the physician delivers the therapeutic agent from the second area 132, 232, 332 through the tubular element 110, 210, 310 into the sub-retinal or suprachoroidal area. Again, this may be done by operating a foot pedal or other remote actuation device. Lower pressure may be used for delivery of the therapeutic agent as compared to the pressure used for bleb formation. When the device 100, 200, 300 is in the second condition, fluid communication between the first area 122, 222, 322 and the tubular element 110, 210, 310 is prevented.

If desired, multiple injections of therapeutic agent may be performed. For example, after a first delivery of therapeutic agent, the tube 230 may be retracted for refilling of the tube with therapeutic agent for a second delivery and as many subsequent deliveries as are desired.

Due to designs as disclosed herein, with only a single needle or microcatheter insertion, the bleb-formation fluid and the therapeutic agent composition may be delivered separately and thus may be delivered at different flow rates and pressures. Thus, the delivery of the bleb-formation fluid into the sub-retinal area (or suprachoroidal area) may be done at higher flow rates and pressure than the delivery of the fragile or delicate therapeutic agent. Higher pressures and flow rates may be required for adequate bleb formation. Lower pressures and flow rates help maintain the integrity and efficacy of delicate therapeutic agents, such as live cell vectors, viral therapeutic agents, gene therapy vectors, and/or stem cells. In some embodiments, the therapeutic agent may be delivered without exposing the therapeutic agent to the higher pressure of the bleb-formation fluid.

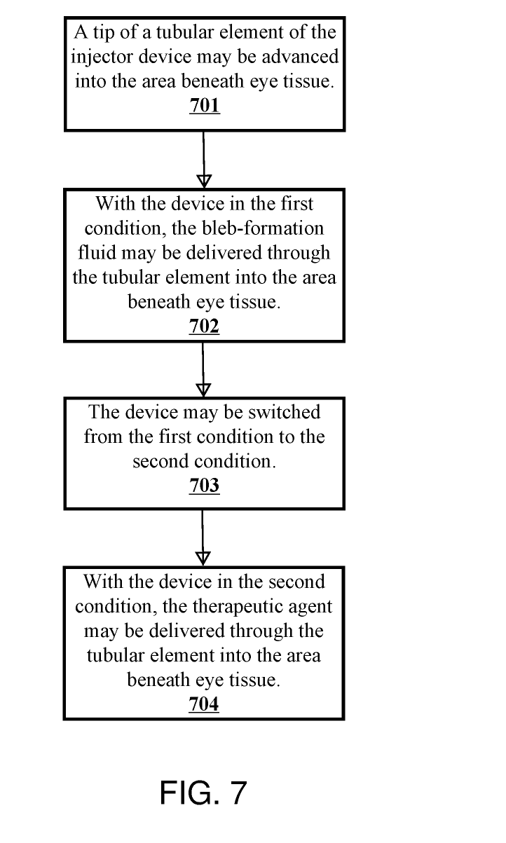

FIG. 7 illustrates a flowchart of a method for delivery of a bleb-formation fluid into an area beneath eye tissue and delivery of a therapeutic agent into that area, according to an embodiment. The elements provided in the flowchart are illustrative only. Various provided elements may be omitted, additional elements may be added, and/or various elements may be performed in a different order than provided below.

At 701, a tip of a tubular element of the injector device may be advanced into the area beneath eye tissue. In some embodiments, the injector device may include: (i) an injector housing having a proximal end and a distal end and an outlet port at the distal end, (ii) the tubular element, connected to the outlet port of the injector housing at the distal end of the injector housing, the tubular element having the tip at a distal end of the tubular element, (iii) a first area within the injector housing configured to be selectively in fluid communication with the tubular element for delivery of a bleb-formation fluid through the tubular element into the area beneath eye tissue for formation of a bleb beneath the eye tissue, and (iv) a second area within the injector housing configured to be selectively in fluid communication with the tubular element for delivery of a therapeutic agent through the tubular element into the area beneath eye tissue at a location of the bleb.

At 702, with the device in a first condition in which the first area is in fluid communication with the tubular element, the bleb-formation fluid may be delivered through the tubular element into the area beneath eye tissue;

At 703, the device may be switched from the first condition to a second condition in which the second area is in fluid communication with the tubular element.

At 704, with the device in the second condition, the therapeutic agent may be delivered through the tubular element into the area beneath eye tissue.

In some embodiments, the method may be performed without removing the tip of the tubular element from the area beneath eye tissue between delivering the bleb-formation fluid and delivering the therapeutic agent. In some embodiments, delivering the bleb-formation fluid and delivering the therapeutic agent may be controlled by one or more actuators remote from the eye that are connected by one or more control lines to the injector device. In some embodiments, delivering the bleb-formation fluid into the area beneath eye tissue may be done at a higher pressure than delivering of the therapeutic agent.

Devices and methods in accordance with certain embodiments as described herein facilitate delivery of a bleb-formation fluid into an area beneath eye tissue and delivery of a therapeutic agent into that area with only one puncture of eye tissue into the desired area and with minimal motion of the tubular element (needle or catheter) throughout the process. Once the tissue is punctured, all actions of the injector can be controlled remotely and can be initiated quickly. In some embodiments, no motion of the hand holding the device is required to actuate the delivery of the bleb-formation fluid and the therapeutic agent composition.

The devices and methods herein facilitate microliter or similarly small doses of therapeutic agent. The devices and methods herein allow precision fluidics control, precision dosage measurement, precision sequential and synchronized fluids delivery, and precision ultra-low pressure control.

Persons of ordinary skill in the art will appreciate that the implementations encompassed by the present disclosure are not limited to the particular exemplary implementations described above. In that regard, although illustrative implementations have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A device for injection of therapeutic agent into an area beneath eye tissue, the device comprising:
an injector housing having a proximal end and a distal end and an outlet port at the distal end;
a tubular element connected to the outlet port of the injector housing at the distal end of the injector housing;
a first area within the injector housing configured to be selectively in fluid communication with the tubular element for delivery of a bleb-formation fluid through the tubular element into the area beneath eye tissue for formation of a bleb in the area beneath eye tissue; and
a second area within the injector housing configured to be selectively in fluid communication with the tubular element for delivery of a therapeutic agent through the tubular element into the area beneath eye tissue at a location of the bleb;
wherein the device comprises a first condition in which the first area is in fluid communication with the tubular element for delivery of the bleb-formation fluid through the tubular element into the area beneath eye tissue and a second condition in which the second area is in fluid communication with the tubular element for delivery of the therapeutic agent through the tubular element into the area beneath eye tissue.

2. The device as in claim 1, wherein when the device is in the first condition, fluid communication between the second area and the tubular element is prevented.

3. The device as in claim 1, wherein when the device is in the second condition, fluid communication between the first area and the tubular element is prevented.

4. The device as in claim 1, further comprising a source of bleb-formation fluid connected to the first area for delivery of the bleb-formation fluid to the first area.

5. The device as in claim 4, further comprising a pressurization mechanism for pressurized delivery of the bleb-formation fluid through the first area and the tubular element and into the area beneath eye tissue.

6. The device as in claim 1, wherein the bleb-formation fluid is a balanced saline solution.

7. The device as in claim 1, further comprising a source of therapeutic agent connected to the second area for delivery of the therapeutic agent to the second area.

8. The device as in claim 7, further comprising a delivery mechanism for low pressure delivery of the therapeutic agent through the second area and the tubular element and into the area beneath eye tissue.

9. The device as in claim 1, wherein the therapeutic agent comprises at least a live cell vector, viral therapeutic agent, a gene therapy vector, or stem cells.

10. The device as in claim 1, wherein the first area is a chamber within the injector housing and the second area is an internal area of a tube within the chamber, wherein the tube is movable between a proximal position in which the tube is away from the outlet port of the injector housing and a distal position in which the tube is adjacent the outlet port of the injector housing, wherein when the tube is in the proximal position the device is in the first condition in which the first area is in fluid communication with the tubular element for delivery of the bleb-formation fluid through the tubular element into the area beneath eye tissue, and when the tube is in the distal position the device is in the second condition in which the second area is in fluid communication with the tubular element for delivery of the therapeutic agent through the tubular element into the area beneath eye tissue.

11. The device as in claim 10, further comprising a valve that seals an outlet end of the tube when the tube is in the proximal position.

12. The device as in claim 10, further comprising a septum that seals an outlet end of the tube when the tube is in the proximal position, wherein the septum is configured to be pierced when the tube is moved to the distal position in order to put the second area in fluid communication with the tubular element.

13. The device as in claim 1, wherein the second area is a chamber within the injector housing and the first area is an internal area of a tube within the chamber, wherein the tube is movable between a proximal position in which the tube is away from the outlet port of the injector housing and a distal position in which the tube is adjacent the outlet port of the injector housing, wherein when the tube is in the distal position the device is in the first condition in which the first area is in fluid communication with the tubular element for delivery of the bleb-formation fluid through the tubular element into the area beneath eye tissue, and when the tube is in the proximal position the device is in the second condition in which the second area is in fluid communication with the tubular element for delivery of the therapeutic agent through the tubular element into the area beneath eye tissue.

14. The device as in claim 1, wherein the tubular element is a needle.

15. The device as in claim 1, wherein the tubular element is a flexible microcatheter.

16. A device for injection of therapeutic agent into an area beneath eye tissue, the device comprising:
an injector housing having a proximal end and a distal end and an outlet port at the distal end;
a tubular element connected to the outlet port of the injector housing at the distal end of the injector housing;
a chamber within the injector housing configured to be selectively in fluid communication with the tubular element for delivery of a bleb-formation fluid through the tubular element into the area beneath eye tissue for formation of a bleb; and
a tube within the injector housing configured to be selectively in fluid communication with the tubular element for delivery of a therapeutic agent through the tubular element into the area beneath eye tissue at a location of the bleb;
wherein the tube is movable between a proximal position in which the tube is away from the outlet port of the injector housing and a distal position in which the tube is adjacent the outlet port of the injector housing, wherein when the tube is in the proximal position the device is in a first condition in which the chamber is in fluid communication with the tubular element for delivery of the bleb-formation fluid through the tubular element into the area beneath eye tissue, and when the tube is in the distal position the device is in the second condition in which the tube is in fluid communication with the tubular element for delivery of the therapeutic agent through the tubular element into the area beneath eye tissue.

17. A method for injection of therapeutic agent into an area beneath eye tissue, the method comprising:
advancing a tip of a tubular element of the injector device into the area beneath eye tissue, the injector device comprising: (i) an injector housing having a proximal end and a distal end and an outlet port at the distal end, (ii) the tubular element, connected to the outlet port of the injector housing at the distal end of the injector housing, the tubular element having the tip at a distal end of the tubular element, (iii) a first area within the injector housing configured to be selectively in fluid communication with the tubular element for delivery of a bleb-formation fluid through the tubular element into the area beneath eye tissue for formation of a bleb beneath the eye tissue, and (iv) a second area within the injector housing configured to be selectively in fluid communication with the tubular element for delivery of a therapeutic agent through the tubular element into the area beneath eye tissue at a location of the bleb;
with the device in a first condition in which the first area is in fluid communication with the tubular element, delivering the bleb-formation fluid through the tubular element into the area beneath eye tissue;
switching the device from the first condition to a second condition in which the second area is in fluid communication with the tubular element; and
with the device in the second condition, delivering the therapeutic agent through the tubular element into the area beneath eye tissue.

18. The method as in claim 17, wherein the method is performed without removing the tip of the tubular element from the area beneath eye tissue between delivering the bleb-formation fluid and delivering the therapeutic agent.

19. The method as in claim 17, wherein delivering the bleb-formation fluid and delivering the therapeutic agent are controlled by one or more actuators remote from the eye that are connected by one or more control lines to the injector device.

20. The method as in claim 17, wherein the delivering of the bleb-formation fluid into the area beneath eye tissue is done at a higher pressure than the delivering of the therapeutic agent.
